(19) United States
(12) Patent Application Publication
(10) Pub. No.: US 2026/0294998 A1
(43) Pub. Date: Oct. 1, 2026

(54) Plant-based amyloid fibrils, their Preparation Method, Composite Film, and Composite Coating Preservative

(71) Applicant: ZHEJIANG UNIVERSITY, Hangzhou (CN)

(72) Inventors: Wenjun WANG, Hangzhou (CN); Tiange PAN, Hangzhou (CN); Hao WANG, Hangzhou (CN); Congyi XU, Hangzhou (CN); Haiqi CHEN, Hangzhou (CN); Kai FU, Hangzhou (CN); Lifen ZHANG, Hangzhou (CN); Ruiling LV, Hangzhou (CN); Songfeng YU, Hangzhou (CN); Jianwei ZHOU, Hangzhou (CN); Donghong LIU, Hangzhou (CN)

(73) Assignee: ZHEJIANG UNIVERSITY, Hangzhou (CN)

(21) Appl. No.: 19/540,593

(22) Filed: Feb. 14, 2026

(30) Foreign Application Priority Data
Mar. 27, 2025 (CN) 2025103754973

(51) Int. Cl. A61K 38/00

(52) U.S. Cl. CPC A61K 38/00; A61K 2300/00

(57) ABSTRACT

A method for preparing plant-based amyloid fibrils includes mixing a plant protein isolate with water, adjusting the pH to 1.5-2.5, and then performing solid-liquid separation. The resulting liquid material containing the plant protein isolate is subjected to a fibrillization treatment to obtain the plant-based amyloid fibrils. According to the growth kinetics curve, the fibrillization treatment sequentially includes a first incubation treatment, an ultrasonic treatment, and a second incubation treatment. The duration of the first incubation ranges from 0 h to the end of the exponential phase, and both the first and second incubation treatments are carried out under non-ultrasonic conditions. When the duration of the first incubation treatment is 0 h, the power density of the ultrasonic treatment is ≥2.5 W/mL. The method enables simultaneous regulation of the fibril growth process and morphology, and reduces the proportion of rigid fibrils.

### REFERENCE TO RELATED APPLICATIONS

This is an application claiming the benefit of priority to a Chinese Patent Application No. 202510375497.3, filed on Mar. 27, 2025, entitled “Plant-based amyloid fibrils, Their Preparation Method, Composite Film and Composite Coating Preservative”, the disclosure of which is incorporated herein by reference in its entirety, including any appendices and attachments thereof, for all purposes.

### FIELD OF TECHNOLOGY

The present disclosure relates to the technical field of protein fibrils, particularly to plant-based amyloid fibrils, their preparation method, a composite film, and a composite coating preservative.

### BACKGROUND

In recent years, the constrained supply of animal proteins has heightened interest in plant-based alternatives, owing to their wide availability. Pea protein isolate, a by-product of pea starch extraction, is currently the second largest protein source among plants after soy, possessing high nutritional value and low allergenicity. Pea protein isolate primarily exists in a globular form. When the globular structure undergoes self-assembly into a high-aspect-ratio fibrillar structure to form pea protein-based amyloid fibrils, the application scenarios of pea protein isolate can be significantly broadened.

The conventional methods typically involve heating globular plant proteins to induce self-assembly to transform into fibrillar structures. However, protein fibrils prepared by these methods often contain an undesirably high proportion of rigid fibrils.

### SUMMARY

The present disclosure provides plant-based amyloid fibrils, their preparation method, a composite film, and a composite coating preservative. The preparation method of the present disclosure facilitates simultaneous regulation of the growth process and morphology of plant-based amyloid fibrils, thereby reducing the proportion of rigid fibrils. The composite film prepared using the plant-based amyloid fibrils exhibits superior mechanical properties, and the composite coating preservative prepared using the plant-based amyloid fibrils exhibits excellent preservation performance.

The present disclosure provides the following technical solutions.

The present disclosure provides a method for preparing plant-based amyloid fibrils, including:
1) mixing a plant protein isolate with water to obtain an aqueous solution of the plant protein isolate;
2) adjusting the pH of the aqueous solution of the plant protein isolate to 1.5-2.5, followed by solid-liquid separation to obtain a liquid material containing the plant protein isolate; and
3) subjecting the liquid material containing the plant protein isolate to a fibrillization treatment to obtain plant-based amyloid fibrils.

According to a growth kinetics curve of the plant-based amyloid fibrils, the fibril growth sequentially includes a lag phase, an exponential phase, and a saturation phase.

The fibrillization treatment sequentially includes a first incubation treatment, an ultrasonic treatment, and a second incubation treatment. The duration of the first incubation treatment ranges from 0 h to the end of the exponential phase, and both the first and second incubation treatments are carried out under non-ultrasonic conditions. When the duration of the first incubation treatment is 0 h, the ultrasonic power density of the ultrasonic treatment is ≥2.5 W/mL.

In some embodiments, the plant protein isolate includes pea protein isolate, and a ratio of plant protein isolate to water is in a range of 1 g:100 mL to 3 g:100 mL.

In some embodiments, the ultrasonic treatment is performed at a temperature of 0-4° C. for a total duration of 1-3 minutes, with an interval of 1-3 seconds after every 1-3 seconds of ultrasonic irradiation. When the duration of the first incubation treatment is 0 h, the ultrasonic power density of the ultrasonic treatment is 2.5-4 W/mL. When the duration of the first incubation treatment is not 0 h, the ultrasonic power density of the ultrasonic treatment is 2-4 W/mL.

In some embodiments, the temperatures of the first and second incubation treatments are independently in a range of 75-85° C., and the total duration of the first and second incubation treatments is in a range of 45-50 h. The first and second incubation treatments are carried out under stirring conditions.

In some embodiments, the duration of the first incubation treatment is in a range of 0 h to the end of the lag phase.

In some embodiments, with the onset of fibril growth defined as t=0, the lag phase spans 0-5 h, and the exponential phase spans 5-26.5 h.

In some embodiments, the fibrillization treatment includes any one of the following:
1) the duration of the first incubation treatment is 0 h, and the ultrasonic power density of the ultrasonic treatment is in a range of 2.6-4 W/mL;
2) the duration of the first incubation treatment is in a range of 4-5 h, and the ultrasonic power density of the ultrasonic treatment is in a range of 3-4 W/mL; and
3) the duration of the first incubation treatment is in a range of 25-26.5 h, and ultrasonic the power density of the ultrasonic treatment is in a range of 2-2.2 W/mL.

The plant-based amyloid fibrils prepared by the method of the present disclosure include flexible fibrils, semi-flexible fibrils, and rigid fibrils. In the plant-based amyloid fibrils of the present disclosure, the proportion of flexible fibrils is in a range of 4-50%, the proportion of semi-flexible fibrils is in a range of 10-45%, and the proportion of rigid fibrils is in a range of 35-65.8%.

The present disclosure also provides a composite film including a chitosan matrix and plant-based amyloid fibrils prepared by the method of the present disclosure. The plant-based amyloid fibrils are distributed within the chitosan matrix.

The present disclosure further provides a composite coating preservative including chitosan, plant-based amyloid fibrils prepared by the method of the present disclosure, epigallocatechin gallate (EGCG), and a solvent.

The method for preparing plant-based amyloid fibrils of the present disclosure facilitates simultaneous regulation of the growth process and morphology of plant-based amyloid fibrils. Specifically, the ultrasonic treatment can accelerate the growth of plant-based amyloid fibrils, which is of significant importance for the production process of fibrils which is conventionally energy-intensive, as it enhances production efficiency and reduces energy consumption. Moreover, the ultrasonic treatment can regulate the morphology of the plant-based amyloid fibrils and reduce the proportion of rigid fibrils. Test results show that the ultrasonic treatment increases the proportion of flexible and semi-flexible fibrils in pea protein-based amyloid fibrils. Composite films prepared using the plant-based amyloid fibrils of the present disclosure exhibit superior mechanical properties, and composite coating preservatives prepared using the plant-based amyloid fibrils exhibit excellent preservation performance. Test results also indicate that the composite coating preservative has an improved inhibitory effect on browning of fresh-cut apples, particularly when the content of flexible and semi-flexible fibrils is relatively high.

### BRIEF DESCRIPTION OF THE DRAWINGS

FIG. 1 is a diagram illustrating the preparation processes for pea protein-based amyloid fibrils with or without ultrasonic treatments, respectively.

FIGS. 2A-2C show the growth kinetics test results of pea protein-based amyloid fibrils under ultrasonic treatment at t0. FIG. 2A shows the normalized growth kinetics curves of pea protein-based amyloid fibrils under ultrasonic treatment at t0. FIG. 2B shows the kinetic parameters of samples treated at to under different ultrasonic intensities, including the lag phase duration, t_{1/2}, and t_{growth}, where t_{1/2} represents the time required to reach half of the maximum ThT fluorescence intensity, and t_{growth} represents the duration of the exponential phase during the fibrillization process. FIG. 2C shows the maximum Thioflavin T (ThT) fluorescence intensity of pea protein-based amyloid fibrils treated at t0 under different ultrasonic intensities.

FIGS. 3A-3C show the growth kinetics test results of pea protein-based amyloid fibrils under ultrasonic treatment at t0.1. FIG. 3A shows the normalized growth kinetics curves of pea protein-based amyloid fibrils under ultrasonic treatment at t0.1. FIG. 3B shows the kinetic parameters of samples treated at t0.1 under different ultrasonic intensities, including the lag phase duration, t_{1/2}, and t_{growth}, where t_{1/2} represents the time required to reach half of the maximum ThT fluorescence intensity, and t_{growth} represents the duration of the exponential phase during the fibrillization process. FIG. 3C shows the maximum ThT fluorescence intensity of pea protein-based amyloid fibrils treated at t0.1 under different ultrasonic intensities.

FIGS. 4A-4B show the growth kinetics test results of pea protein-based amyloid fibrils under ultrasonic treatment at t0.9. FIG. 4A shows the normalized growth kinetics curves of pea protein-based amyloid fibrils under ultrasonic treatment at t0.9. FIG. 4B shows the maximum Thioflavin T (ThT) fluorescence intensity of pea protein-based amyloid fibrils treated at t0.9 under different ultrasonic intensities.

FIG. 5 is a TEM image of pea protein-based amyloid fibril samples.

FIGS. 6A-6D show the contour length distributions of pea protein-based amyloid fibrils under ultrasonic treatment. FIG. 6A shows the contour length distribution of fibril samples with different heating durations. FIG. 6B shows the contour length distribution of fibrils in samples that were subjected to ultrasonic treatment at the beginning of the lag phase (0 h) and subsequently incubated. FIG. 6C shows the contour length distribution of fibrils in samples that were subjected to at the end of the lag phase (5 h) and subsequently incubated. FIG. 6D shows the contour length distribution of fibrils in samples that were subjected to ultrasonic treatment at the end of the exponential phase (26.5 h) and subsequently incubated.

FIG. 7 is a statistical diagram of the proportions of flexible, semi-flexible, and rigid fibrils in pea protein-based amyloid fibrils under ultrasonic treatment at t0, t0.1, and t0.9.

FIG. 8 is a diagram showing tensile strength and elongation at break of the composite films of the present disclosure.

FIG. 9 is a diagram showing the correlation between the mechanical properties and fibril rigidity/flexibility of the composite films of the present disclosure.

FIG. 10 is a diagram showing the DPPH radical scavenging activity of pea protein isolate and pea protein-based amyloid fibrils.

FIG. 11 is a diagram showing the preservation performance of fresh-cut apples coated with the composite coating preservative of the present disclosure.

### DETAILED DESCRIPTION

The present disclosure provides a method for preparing plant-based amyloid fibrils, including:
1) mixing a plant protein isolate with water to obtain an aqueous solution of the plant protein isolate;
2) adjusting the pH of the aqueous solution of the plant protein isolate to 1.5-2.5, followed by solid-liquid separation to obtain a liquid material containing the plant protein isolate; and
3) subjecting the liquid material containing the plant protein isolate to a fibrillization treatment to obtain plant-based amyloid fibrils.

According to a growth kinetics curve of the plant-based amyloid fibrils, the fibril growth sequentially includes a lag phase, an exponential phase, and a saturation phase.

The fibrillization treatment sequentially includes a first incubation treatment, an ultrasonic treatment, and a second incubation treatment. The duration of the first incubation treatment ranges from 0 h to the end of the exponential phase, and both the first and second incubation treatments are carried out under non-ultrasonic conditions. When the duration of the first incubation treatment is 0 h, the ultrasonic power density of the ultrasonic treatment is ≥2.5 W/mL.

The present disclosure facilitates simultaneous regulation of both the growth process and morphology of plant-based amyloid fibrils through ultrasonic treatment. Specifically, ultrasound is an efficient, low-energy, and environmentally friendly physical processing technique that can alter the various hierarchical structures of proteins through external energy input without introducing exogenous substances, so as to improve protein functional properties. In the present disclosure, ultrasound is applied at different stages during the growth of plant-based amyloid fibril (e.g., the initial stage of the growth is designated as to, the end of the lag phase is designated as t0.1, and the end of the growth phase is designated as t0.9) to provide flexible control over the timing of regulation. Furthermore, the rigidity/flexibility indices illustrating the morphology of the plant-based amyloid fibril are quantified, allowing the correlation between fibril morphology and function to be established. Based on this, the performance differences of plant-based amyloid fibrils with different morphologies in protein-chitosan composite film systems and composite coating preservatives are further investigated. Plant-based amyloid fibril samples with higher contents of flexible and semi-flexible fibrils exhibit superior mechanical properties, antioxidant performance, and preservation performance. The following provides a detailed description of the method for preparing plant-based amyloid fibrils according to the present disclosure.

Unless otherwise specified, all raw materials used in the present disclosure are commercially available products familiar to those skilled in the art or are prepared using methods well-known to those skilled in the art.

In the present disclosure, a plant protein isolate is mixed with water to obtain an aqueous solution of the plant protein isolate. In some embodiments, the plant protein isolate may include pea protein isolate. A method for preparing the pea protein isolate may include the following steps: mixing pea protein powder with water and performing a first stirring treatment to obtain a first mixture; adjusting the pH of the first mixture to 7.5-8.5 and performing a second stirring treatment; performing a first solid-liquid separation to obtain a liquid material; adjusting the pH of the liquid material to 4-5 and performing a third stirring treatment; performing a second solid-liquid separation to obtain a solid material; mixing the solid material with water to form a second mixture, adjusting the pH of the second mixture to 6.5-7.5 under stirring until the solid material is completely dissolved, to obtain a protein solution, performing dialysis on the protein solution to obtain a purified material, and freeze-drying the purified material to obtain the pea protein isolate.

In some embodiments, when mixing the pea protein powder with water, the mass-to-volume ratio of the pea protein powder to water is in a range of 0.5-1.5 g:10 mL, preferably 1 g:10 mL. The duration of the first stirring treatment may be in a range of 1.5-2.5 h, preferably 2 h. The reagent used for adjusting the pH of the first mixture to 7.5-8.5 (preferably 8.0) may be a NaOH solution, with a concentration of 3 M. The duration of the second stirring treatment may be in a range of 1.5-2.5 h, preferably 2 h.

In some embodiments, the first and second solid-liquid separations may be performed by centrifugation; the centrifugation temperature may be in a range of 3-6° C., preferably 4° C.; the rotational speed of the centrifugation may be in a range of 6,000-10,000 rpm, preferably 8,000 rpm; and the duration of centrifugation may be in a range of 15-25 min, preferably 20 min.

In some embodiments, the reagent used for adjusting the pH of the liquid material to 4-5 (preferably 4.5) may be hydrochloric acid with a concentration of 3 M. The duration of the third stirring treatment may be in a range of 0.5-1.5 h, preferably 1 h. After the second solid-liquid separation, when the obtained solid material is mixed with water, the mass-to-volume ratio of the solid material to water may be in a range of 0.5-1.5 g:5-15 mL, preferably 1 g:10 Ml. The reagent used for adjusting the pH of the second mixture to 6.5-7.5 (preferably 7.0) may be a NaOH solution with a concentration of 3 M.

In some embodiments, the dialysis may be performed by a dialysis bag; a molecular weight cut-off of the dialysis bag may be in a range of 8-14 kDa; the dialysis solution may be water, preferably deionized water; the dialysis temperature may be in a range of 3-6° C., preferably 4° C.; and the duration of the dialysis may be in a range of 42-54 h, preferably 48 h. During dialysis, it is preferable to replace the dialysis solution every 6 h. The freeze-drying step of the present disclosure may be performed under conditions well known to those skilled in the art.

After preparing the plant protein isolate (e.g., pea protein isolate), it is mixed with water to obtain an aqueous solution of the plant protein isolate. In some embodiments, the mass-to-volume ratio of the plant protein isolate to water may be in a range of 1-3 g:100 mL, preferably 2 g:100 mL. The method for mixing the plant protein isolate with water is not limited, provided that the plant protein isolate is fully dissolved in water.

After obtaining the aqueous solution of the plant protein isolate, the pH of the aqueous solution of the plant protein isolate is adjusted to 1.5-2.5, followed by solid-liquid separation to obtain a liquid material containing the plant protein isolate. In one embodiment, the pH of the aqueous solution of the plant protein isolate is adjusted to 1.5-2.5 (preferably 2.0) using a reagent such as hydrochloric acid, with a concentration of 3 M. Adjusting the pH to the range of 1.5-2.5 allows for acid hydrolysis of the plant protein isolate during subsequent incubation, which is conducive to the preparation of plant-based amyloid fibrils. In one embodiment, the solid-liquid separation may be performed by centrifugation; the centrifugation temperature may be in a range of 3-6° C., preferably 4° C.; the rotational speed of the centrifugation may be in a range of 6,000-10,000 rpm, preferably 8,000 rpm; and the duration of the centrifugation may be in a range of 15-25 min, preferably 20 min.

After obtaining the liquid material containing the plant protein isolate, it undergoes a fibrillization treatment to obtain the plant-based amyloid fibrils. In some embodiments, the plant-based amyloid fibrils may include pea protein-based amyloid fibrils. In the embodiments of the present disclosure, pea protein isolate is taken as an example, and pea protein-based amyloid fibrils are ultimately prepared.

In the present disclosure, according to the growth kinetics curve of the plant-based amyloid fibrils, the fibril growth during the fibrillization treatment sequentially includes a lag phase, an exponential phase, and a saturation phase. The lag phase specifically refers to the initial stage of amyloid fibril growth, during which the protein in native conformation is hydrolyzed into protein monomers or short peptides under high-temperature and highly acidic conditions. These protein monomers can aggregate via non-covalent interactions to form oligomers rich in cross-β sheet structures, which corresponds to the nucleation process during fibril growth. During the self-assembly of protein monomers into oligomers, Thioflavin T (ThT), a fluorescent dye that specifically binds to the cross-β sheet structures of amyloid fibrils, is used for characterization. It is generally considered that 0-10% of the maximum fluorescence intensity corresponds to the lag phase of amyloid fibril growth.

The exponential phase specifically refers to the stage of rapid fibril growth, during which once the oligomers reach a certain size, they serve as templates, protein monomers self-assemble onto the ends of the oligomers through IT-TT interactions and hydrophobic interactions to form protofibrils with high aspect ratios. It is generally considered that 10-90% of the maximum fluorescence intensity corresponds to the exponential phase.

The saturation phase specifically refers to the mature stage of amyloid fibril growth, during which the concentration of protein monomers in the system is no longer sufficient to support rapid self-assembly. The growth rate slows down, and the protofibrils begin to interact with each other, self-assembling into mature amyloid fibrils through entanglement and ordered arrangements. It is generally considered that above 90% of the maximum fluorescence intensity corresponds to the saturation phase. The method for obtaining the growth kinetics curve of pea protein-based amyloid fibrils is described in detail elsewhere in the present disclosure and will not be repeated here. In the embodiments of the present disclosure, pea protein-based amyloid fibrils are taken as an example, according to their growth kinetics curve, with the onset of fibril growth defined as t=0, the lag phase spans 0-5 h, the exponential phase spans 5-26.5 h, and any timepoint beyond 26.5 h corresponds to the saturation phase.

In the present disclosure, the fibrillization treatment sequentially includes a first incubation treatment, an ultrasonic treatment, and a second incubation treatment. The duration of the first incubation treatment ranges from 0 h to the end of the exponential phase, and both the first and second incubation treatments are performed under non-ultrasonic conditions.

In some embodiment, the duration of the first incubation treatment may be 0 h to the end of the exponential phase. In some embodiments, the duration of the first incubation treatment may further be 0 h to the end of the lag phase, preferably 0 h (i.e., the first incubation treatment is skipped, and the ultrasonic treatment is performed directly). In some embodiments, the temperature of the first incubation treatment may be in a range of 75-85° C., preferably 80° C. The first incubation treatment may be performed under stirring conditions, with a stirring speed of 350-450 rpm, preferably 400 rpm.

In some embodiments, the ultrasonic treatment is performed at a temperature of 0-4° C. (preferably 0-2° C.) for a total duration of 1-3 minutes (preferably 2 min), with an interval of 1-3 seconds (preferably 2 s) after every 1-3 seconds (preferably 2 s) of ultrasonic irradiation. When the duration of the first incubation treatment is 0 h, the ultrasonic power density of the ultrasonic treatment is ≥2.5 W/mL, preferably 2.5-4 W/mL, specifically 2.5 W/mL, 2.6 W/mL, 2.7 W/mL, 3 W/mL, 3.5 W/mL, or 4 W/mL. When the duration of the first incubation treatment is not 0 h, the ultrasonic power density of the ultrasonic treatment is in a range of 2-4 W/mL, specifically 2 W/mL, 2.2 W/mL, 2.4 W/mL, 2.5 W/mL, 2.6 W/mL, 2.7 W/mL, 3 W/mL, 3.5 W/mL, or 4 W/mL.

In the examples of the present disclosure, the diameter of the ultrasonic horn used is 10 mm. In the examples of the present disclosure, the effects of different ultrasonic intensities on plant-based amyloid fibril growth were investigated. The ultrasonic intensity may range from 1-30%, specifically 1%, 5%, 10%, 20%, and 30%, corresponding to actual ultrasonic power densities of 2.1766 W/mL, 2.3909 W/mL, 2.6588 W/mL, 3.1946 W/mL, and 3.7304 W/mL, respectively. In the examples of the present disclosure, the rated power of the ultrasonic device at 100% intensity is 900 W.

In one embodiment, the total duration of the first and second incubation treatments may range from 45-50 h, preferably 48 h. The temperature of the second incubation treatment may range from 75-85° C., preferably 80° C. The second incubation treatment can be performed under stirring conditions with a stirring speed of 350-450 rpm, preferably 400 rpm.

In one embodiment, the fibrillization treatment includes any one of the following three options:
Option 1: The duration of the first incubation treatment is 0 h, and the ultrasonic power density of the ultrasonic treatment is in a range of 2.6-4 W/mL. In the examples of the present disclosure, the liquid material containing the plant protein isolate is sequentially subjected to ultrasonic treatment and the second incubation treatment. The ultrasonic intensity may be 10%, 20%, or 30% (corresponding to actual ultrasonic power densities of 2.6588 W/mL, 3.1946 W/mL, and 3.7304 W/mL). The ultrasonic treatment is performed in an ice-water bath for 2 min, with an interval of 2 seconds after every 2 seconds of ultrasonic irradiation. The second incubation treatment is performed at 80° C. for 48 h under stirring at 400 rpm.
Option 2: The duration of the first incubation treatment is in a range of 4-5 h, and the ultrasonic power density of the ultrasonic treatment is in a range of 3-4 W/mL. In the examples of the present disclosure, the liquid material containing the plant protein isolate is sequentially subjected to the first incubation treatment, the ultrasonic treatment, and the second incubation treatment. The first incubation treatment is conducted at 80° C. for 5 h under stirring at 400 rpm. The ultrasonic intensity may be 20% or 30% (corresponding to actual ultrasonic power densities of 3.1946 W/mL and 3.7304 W/mL). The ultrasonic treatment is performed in an ice-water bath for 2 min, with an interval of 2 seconds after every 2 seconds of ultrasonic irradiation. The second incubation treatment is performed at 80° C. for 43 h under stirring at 400 rpm.
Option 3: The duration of the first incubation treatment is in a range of 25-26.5 h, and the ultrasonic power density of the ultrasonic treatment is in a range of 2-2.2 W/mL. In the examples of the present disclosure, the liquid material containing the plant protein isolate is sequentially subjected to the first incubation treatment, the ultrasonic treatment, and second incubation treatment. The first incubation treatment is conducted at 80° C. for 26.5 h under stirring at 400 rpm. During the ultrasonic treatment, the ultrasonic intensity may be 1% (corresponding to an actual ultrasonic power density of the ultrasonic treatment of 2.1766 W/mL). The ultrasonic treatment is performed in an ice-water bath for 2 min, with an interval of 2 seconds after every 2 seconds of ultrasonic irradiation. The second incubation treatment is conducted at 80° C. for 21.5 h under stirring at 400 rpm.

In the present disclosure, ultrasonic treatment may be applied at different stages during the growth of plant-based amyloid fibril (initial of the growth is designated as t0, end of the lag phase is designated as t0.1, and end of the growth phase is designated as t0.9) using an ultrasonic device with a rated power of 900 W and ultrasonic intensities of 1%, 5%, 10%, 20%, and 30% to control both fibril growth and morphology. Specifically, regarding fibril growth, high-intensity ultrasound (10%, 20%, and 30%) at to significantly accelerates fibrillization without notably reducing fibril yield; high-intensity ultrasound (20% and 30%) at t0.1 significantly increases the growth rate of plant-based amyloid fibrils during the exponential phase; low-intensity ultrasound (1%) at t0.9 can enhance the fibril growth rate. Regarding fibril morphology, high-intensity ultrasound (10%, 20%, and 30%) at to significantly reduces the proportion of rigid fibrils while increasing the proportion of flexible and semi-flexible fibrils; at t0.1, ultrasound with all intensities significantly reduces the proportion of rigid fibrils; at t0.9, ultrasound with all intensities reduces the proportion of rigid fibrils, while the proportion of flexible fibrils increases with increasing ultrasound intensity.

After the second incubation treatment, the resulting product is preferably subjected sequentially to dialysis and freeze-drying to obtain the plant-based amyloid fibrils. In one embodiment, the dialysis is performed by a dialysis bag; a molecular weight cut-off of the dialysis bag may be in a range of 8-14 kDa; the dialysis solution may be an acidic aqueous solution (pH preferably 2.0); the dialysis temperature may be in a range of 3-6° C., preferably 4° C.; and the duration of the dialysis may be in a range of 42-54 h, preferably 48 h. During dialysis, it is preferable to replace the dialysis solution every 6 h. The freeze-drying step of the present disclosure may be performed under conditions well known to those skilled in the art.

The plant-based amyloid fibrils prepared by the method of the present disclosure include flexible fibrils, semi-flexible fibrils, and rigid fibrils. The percentage of flexible fibrils in the plant-based amyloid fibrils is in a range of 4-50%, preferably 10-45%, and more preferably 20-40%. In the examples, a specific percentage of the flexible fibrils may be 4.76%, 4.95%, 6.48%, 5.00%, 10.89%, 13.00%, 15.84%, 16.83%, 25.00%, 31.00%, 36.00%, 40.00%, or 47.00%. The percentage of semi-flexible fibrils in the plant-based amyloid fibrils is in a range of 10-45%, preferably 20-40%, more preferably 30-35%. In the examples, a specific percentage of the semi-flexible fibrils may be 10.00%, 18.00%, 19.00%, 27.78%, 30.00%, 33.00%, 33.66%, 34.65%, 35.00%, 39.60%, 40.00%, 40.59%, or 43.00%. The percentage of rigid fibrils in the plant-based amyloid fibrils is in a range of 35-65.8%, preferably 40-56%, more preferably 45-50%. In the examples, a specific percentage of the rigid fibrils may be 35.00%, 39.00%, 42.00%, 43.56%, 45.00%, 49.51%, 50.00%, 52.00%, 54.46%, 55.24%, 55.45%, or 65.74%.

The present disclosure provides a composite film including a chitosan matrix and plant-based amyloid fibrils, which are prepared by the method of the present disclosure, wherein the plant-based amyloid fibrils are distributed within the chitosan matrix. In some embodiments, the mass ratio of the chitosan matrix to the plant-based amyloid fibrils in the composite film may be in a range of 2-4:1, preferably 3:1. The thickness of the composite film may be in a range of 0.140-0.160 mm. The composite film of the present disclosure can be used for the preservation of fresh-cut fruits and vegetables.

In one embodiment, the method for preparing the composite film includes: mixing a chitosan solution, a dispersion of plant-based amyloid fibrils, and glycerol to obtain a film-forming solution; placing the film-forming solution in a mold and performing drying to remove the solvent, to obtain the composite film.

In one embodiment, the chitosan solution may be prepared by the following steps: mixing chitosan with an acetic acid solution (volume concentration 2%) at a ratio of 2-4 g:100 mL, preferably 3 g:100 mL, and performing stirring until the chitosan is completely dissolved; adjusting the pH of the mixture to 1.8-2.0 using hydrochloric acid (with a concentration of 3 M) to obtain the chitosan solution. In one embodiment, the dispersion of plant-based amyloid fibrils may be prepared by mixing plant-based amyloid fibrils with water at a ratio of 0.8-1.2 g:100 mL, preferably 1 g:100 mL. In one embodiment, the volume ratio of the chitosan solution, the dispersion of plant-based amyloid fibrils, and glycerol may be in a range of 15: (13-17): (0.3-0.7), preferably 15:15:0.5. In the examples of the present disclosure, the mold may preferably be a petri dish with a diameter of 9 cm. The drying temperature may be in a range of 45-55° C., preferably 50° C.; and the drying duration may be in a range of 15-20 h, preferably 18 h. In the examples of the present disclosure, after drying, the resulting film is placed in a desiccator to equilibrate for 24 h to obtain the composite film.

The present disclosure also provides a composite coating preservative including chitosan, plant-based amyloid fibrils prepared by the method of the present disclosure, epigallocatechin gallate (EGCG), and a solvent.

In one embodiment, the mass ratio of chitosan, plant-based amyloid fibrils, and EGCG may be in a range of (2-4): (0.5-1.5): (1-3), preferably 3:1:2. In one embodiment, the concentration of plant-based amyloid fibrils in the composite coating preservative may be in a range of 8-12 mg/mL, preferably 10 mg/mL. In one embodiment, the pH of the composite coating preservative may be in a range of 1.8-2.0. The solvent is water. The composite coating preservative of the present disclosure can be used for the preservation of fresh-cut fruits and vegetables, for example, fresh-cut apples. Test results of the present disclosure show that fibril groups with higher contents of flexible and semi-flexible fibrils (t0.1 10%; t0.9 30%) exhibit the best browning inhibition effect, with b* values significantly lower than those of the original protein isolate and protein fibrils obtained without ultrasonic treatment.

The present disclosure does not impose specific limitations on the method for preparing the composite coating preservative; those skilled in the art may adopt other methods to prepare the composite coating preservative, provided that all components are uniformly mixed. In the examples of the present disclosure, the film-forming solution may be prepared according to the above method, and then mixed with an EGCG solution to obtain the composite coating preservative. The pH of the EGCG solution may be in a range of 1.8-2.0, and the concentration and volume of the EGCG solution may be adjusted to ensure that the concentration of each component in the resulting composite coating preservative meet the aforementioned requirements.

In the examples of the present disclosure, pea protein isolate is used as an example to study the effects of ultrasonic power density and timing of the ultrasonic treatment during the fibrillization process on the growth kinetics and the final morphology of pea protein-based amyloid fibrils. The physicochemical properties and structural differences of the original protein isolate and amyloid fibrils before and after the ultrasonic treatment were analyzed, revealing the mechanism by which ultrasound regulates pea protein isolate fibrillization. Furthermore, the performance differences of plant-based amyloid fibrils with different morphologies in protein-chitosan composite film and composite coating preservatives were explored.

The following description, in conjunction with the examples of the present disclosure, provides a clear and complete explanation of the present disclosure. The described examples represent only a part of the embodiments of the present disclosure, not all embodiments. Based on the examples described herein, all other examples that can be obtained by those skilled in the art without creative effort also fall within the scope of the present disclosure.

In the following experiments, pea protein isolate was prepared as follows:

Pea protein powder (purchased from Xi'an Virgin Biological Technology Co., Ltd., purity 80 wt %) was mixed with deionized water at a ratio of 1 g:10 mL and stirred for 2 h. The pH of the mixture was then adjusted to 8.0 using 3 M NaOH solution, followed by stirring for 2 h to ensure complete dissolution of the pea protein powder. The resulting solution was centrifuged at 8000 rpm and 4° C. for 20 min, and the supernatant was collected. The pH of the supernatant was adjusted to 4.5 using 3 M HCl and stirred for 1 h, followed by centrifugation at 8000 rpm and 4° C. for 10 min to collect the precipitate. The precipitate was mixed with deionized water at a ratio of 1 g:10 mL to form a mixture, and under continuous stirring, the pH of the mixture was adjusted to 7.0 using 3 M NaOH solution. After the precipitate was completely dissolved, the resulting protein solution was dialyzed at 4° C. for 48 h using a dialysis bag with a molecular weight cut-off of 8-14 kDa, to obtain a purified material. Deionized water was used as the dialysis solution, which was replaced every 6 h. The purified material was finally freeze-dried to obtain the pea protein isolate.

In the following experiments, the method for acquiring the growth kinetic curves of pea protein-based amyloid fibrils and the method for determining t0.1 and t0.9 include the following steps.

Pea protein isolate was mixed with deionized water at a ratio of 2 g:100 mL and stirred at 25° C. for 2 h to fully dissolve the pea protein isolate, to obtain a 2% (w/v) pea protein isolate solution. The pH of the pea protein isolate solution was adjusted to 2.0 using 3 M HCl, followed by centrifugation at 8000 rpm and 4° C. for 10 min to collect the supernatant. The supernatant was incubated in a water bath at 80° C. under stirring at 400 rpm for 48 h. During the incubation, samples were collected at predetermined time intervals. For each sample, 15 μL was withdrawn for subsequent Thioflavin T (ThT) fluorescence measurement.

8.0 mg of ThT powder was added to 10 ml of phosphate-buffered saline (PBS, pH 7.0) containing 150 mM NaCl and stirred for 20 min. The resulting solution was filtered through a 0.22 μm microporous membrane to obtain a ThT stock solution (0.08%, w/v). The ThT stock solution was stored at 4° C. in the dark to prevent degradation. The ThT stock solution was diluted 50-fold with PBS to obtain a ThT working solution.

Each 15 μL sample collected at predetermined time intervals during incubation was mixed with 135 μL of acidic aqueous solution (pH 2.0), and 2850 μL of ThT working solution was then added. The mixture was incubated in the dark at 25° C. for 10 min, and the ThT fluorescence intensity was measured using a microplate reader at an excitation wavelength of 460 nm and an emission wavelength of 490 nm. The ThT fluorescence intensity data (incubation time and fluorescence intensity) were analyzed using the AmyloFit website (http://www.amylofit.ch.cam.ac.uk) to generate the growth kinetics curve of pea protein-based amyloid fibrils. Based on the obtained growth kinetics curve, the time points corresponding to the three stages of fibril growth were determined: the lag phase spans from time zero to the time point corresponding to 10% of the maximum fluorescence intensity, the exponential phase spans from the time point corresponding to 10% of the maximum fluorescence intensity to the time point corresponding to 90% of the maximum fluorescence intensity, and the saturation phase spans from the time point corresponding to 90% of the maximum fluorescence intensity to the end of the incubation. The time corresponding to 10% of the maximum fluorescence intensity is denoted as t0.1, and the time corresponding to 90% of the maximum fluorescence intensity is denoted as t0.9. After these time points were determined, ultrasonic treatment was performed on intermediate products of pea protein-based amyloid fibrils in Example 1 according to the relevant time points.

### Example 1

Pea protein-based amyloid fibrils were prepared as follows. Pea protein isolate was mixed with deionized water at a ratio of 2 g:100 mL. Magnetic stirring was performed at 25° C. for 2 h until full dissolution was achieved, to obtain a 2% (w/v) pea protein isolate solution. The pH of the pea protein isolate solution was adjusted to 2.0 using 3 M HCl. Centrifugation was performed at 8000 rpm and 4° C. for 10 min to collect the supernatant. The supernatant was placed in a water bath at 80° C., and the first incubation treatment was conducted at 0 h (t0), 5 h (end of the lag phase, t0.1), and 26.5 h (end of the exponential phase, t0.9) under stirring at 400 rpm. Each sample (10 mL) collected after the first incubation treatment was then immersed in an ice-water bath and subjected to ultrasonic treatment. Ultrasonic treatment was performed using a 10 mm diameter horn, and for each group of samples subjected to ultrasonication, the ultrasonic intensities were set to 1%, 5%, 10%, 20%, and 30% (with the ultrasonic intensity defined as 100%, the rated power of the ultrasonic device is 900 W). The actual ultrasonic power densities corresponding to these ultrasonic intensities are listed in Table 1. The ultrasonic treatment was performed for a total duration of 2 minutes, with an interval of 2 seconds after every 2 seconds of ultrasonic irradiation. The treated samples were labeled as t0 1-30% group, t0.1 1-30% group, and t0.9 1-30% group. After the ultrasonic treatment, the samples were placed in the 80° C. water bath and subjected to a second incubation under stirring at 400 rpm to obtain pea protein-based amyloid fibrils. The total duration of the first and second incubation treatments was 48 h. During the second incubation, samples were taken at predetermined intervals, and Thioflavin T (ThT) fluorescence intensity was measured as described above to obtain the growth kinetics curve of the pea protein-based amyloid fibrils under ultrasonic treatment.

TABLE 1
Correspondence between Ultrasonic Intensity and
Actual Ultrasonic Power Density of the Ultrasonic
Treatment (measured by isothermal calorimetry)

| Ultrasonic | Actual Ultrasonic Power |
|---|---|
| Intensity | density of the Ultrasonic |
| (%) | Treatment (W/mL) |
| 1 | 2.1766 |
| 5 | 2.3909 |
| 10 | 2.6588 |
| 20 | 3.1946 |
| 30 | 3.7304 |

FIG. 1 is a diagram illustrating the preparation processes for pea protein-based amyloid fibrils with and without ultrasonic treatments, respectively. The lower part of FIG. 1 illustrates the process using ultrasonic treatment, while the upper part of FIG. 1 illustrates the process without ultrasonic treatment. In FIG. 1, “PPI” represents pea protein isolate; “control t0,” “control t0.1,” “control t0.9,” and “control 48 h (t0.9 F)” represent samples of PPI incubated at 80° C. for 0 h, 5 h, 26.5 h, and 48 h, respectively. The time points were determined according to the growth kinetics curve of pea protein-based amyloid fibrils. “10 (1-30%) U,” “t0.1 (1-30%) U,” and “t0.9 (1-30%) U” represent PPI samples that were subjected to ultrasonic treatment after incubation at 80° C. for 0 h, 5 h, and 26.5 h, respectively, with ultrasonic intensities of 1%, 5%, 10%, 20%, and 30%. “t0 (1-30%) F,” “t0.1 (1-30%) F,” and “t0.9 (1-30%) F” represent the final pea protein-based amyloid fibrils obtained from t0 (1-30%) U, t0.1 (1-30%) U, and t0.9 (1-30%) U, respectively. To better reflect practical production conditions, as shown in the upper part of FIG. 1, in the process without ultrasonic treatment, the samples corresponding to 80% of the maximum ThT fluorescence intensity were selected as the final pea protein-based amyloid fibrils for the to group and t0.1 group, while the sample after 48 h of incubation was selected as the final pea protein-based amyloid fibrils for the t0.9 group.

### Test Example 1

### 1. Growth Kinetics of Pea Protein-Based Amyloid Fibrils Under Ultrasonic Treatment

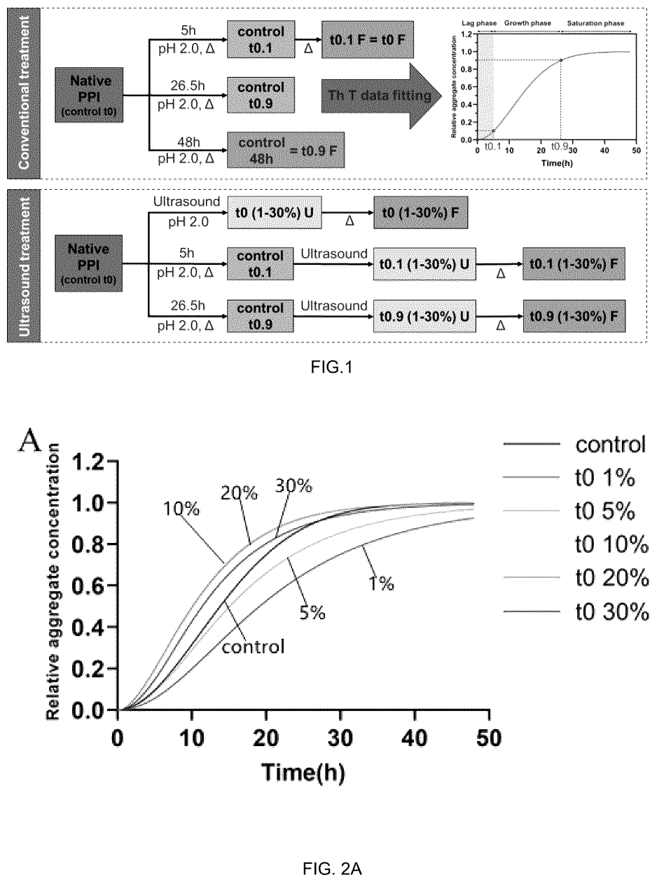

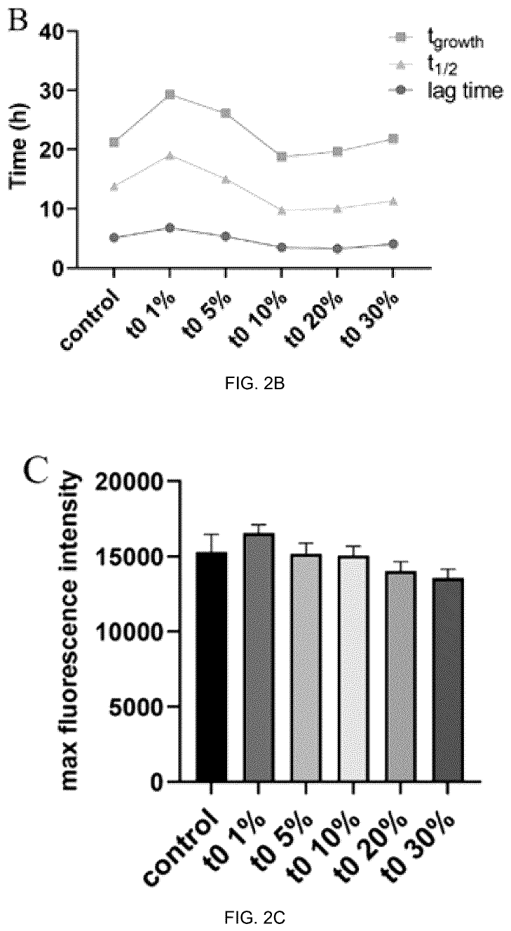

FIGS. 2A-2C show the growth kinetics test results of pea protein-based amyloid fibrils under ultrasonic treatment at t0. FIG. 2A shows the normalized growth kinetics curves of pea protein-based amyloid fibrils under ultrasonic treatment at t0. FIG. 2B shows the kinetic parameters of samples treated at t0 under different ultrasonic intensities, including the lag phase duration, t_{1/2}, and t_{growth}, where t_{1/2} represents the time required to reach half of the maximum ThT fluorescence intensity, and t_{growth} represents the duration of the exponential phase during the fibrillization process. FIG. 2C shows the maximum Thioflavin T (ThT) fluorescence intensity of pea protein-based amyloid fibrils treated at to under different ultrasonic intensities.

The results demonstrated that ultrasonic treatment at to induced significant differences in the fibrillization process of pea protein isolates. Specifically, when the ultrasonic intensity was ≥10% (t0 10%, 20%, and 30%), the fibrillization process was significantly accelerated compared with the control, which corresponded to the shortened lag phase and t_{1/2} observed in FIG. 2B. Conversely, when ultrasonic intensities were 1% or 5%, the fibrillization process was slightly delayed. These indicated that ultrasonic treatment with high power density, performed at to, effectively promoted the fibrillization process. This phenomenon was attributed to that the ultrasonic treatment with high power density promoted the conformational unfolding of pea protein molecules before heating, thereby lowering the energy barrier required for subsequent acid hydrolysis of the protein and facilitating intermolecular self-assembly and nucleation, ultimately accelerating the fibrillization process. As shown in FIG. 2C, the maximum ThT fluorescence intensity of the to group samples slightly decreased with increasing ultrasonic power, but did not differ significantly from the control. This indicated that ultrasonic treatment had a minimal effect on the overall fibrillization extent of pea protein-based amyloid fibrils. Notably, the samples subjected to ultrasonic treatment at t0 with an ultrasonic intensity of 1% (denoted as t0 1%) exhibited a slightly higher maximum fluorescence than the control (P>0.05), suggesting that more fibrils were generated. Taken together, the results indicated that the growth kinetics of pea protein-based amyloid fibrils can be modulated by ultrasonic treatment at varying power densities applied at t0; specifically, the fibrillization process was accelerated by high-power-density ultrasonic treatment without compromising fibril yield.

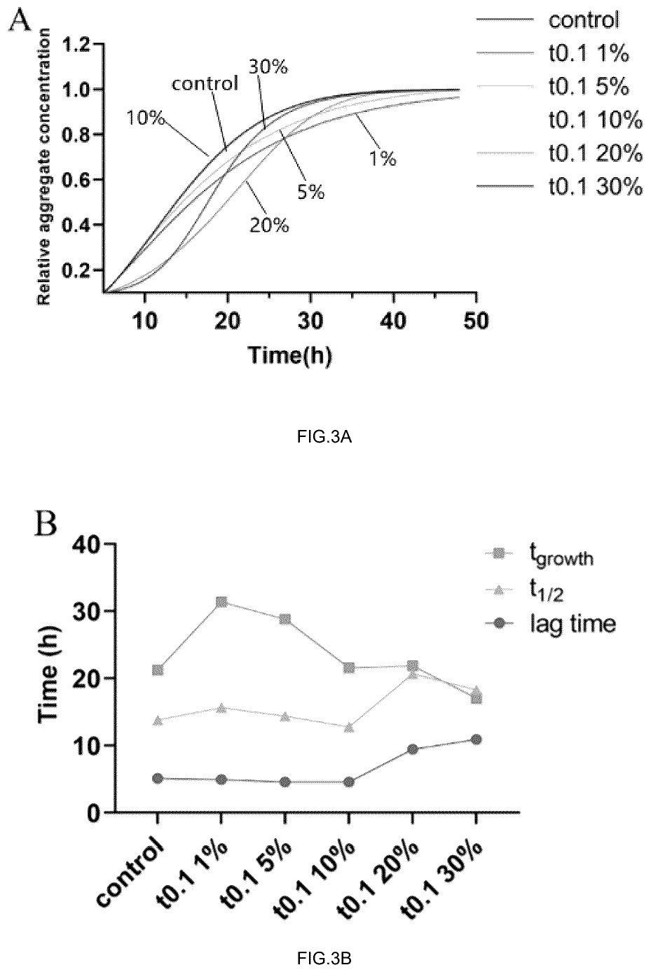

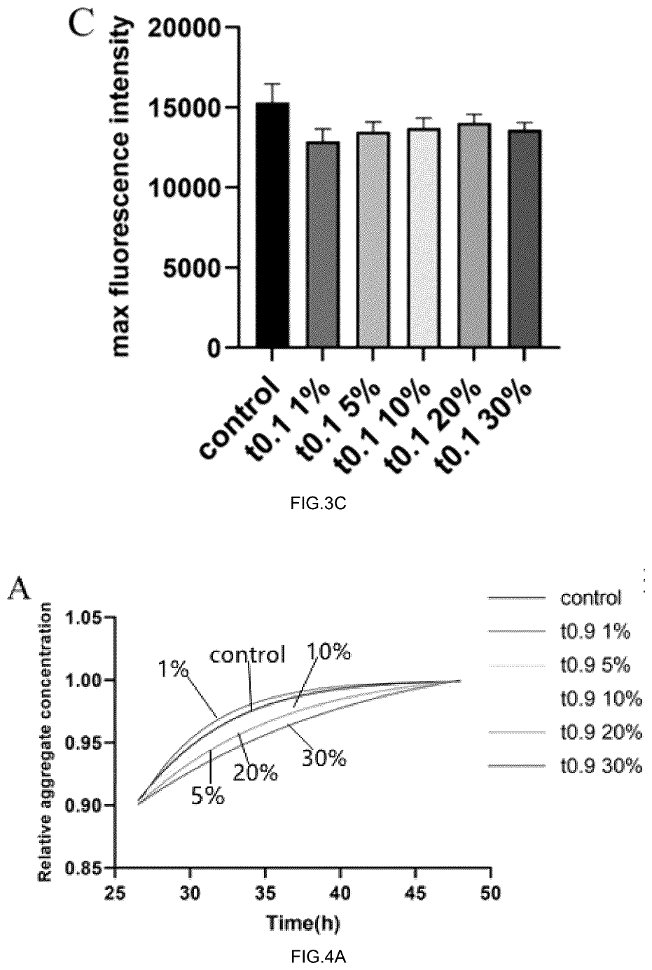

FIGS. 3A-3C show the growth kinetics test results of pea protein-based amyloid fibrils under ultrasonic treatment at t0.1. FIG. 3A shows the normalized growth kinetics curves of pea protein-based amyloid fibrils under ultrasonic treatment at t0.1. FIG. 3B shows the kinetic parameters of samples treated at t0.1 under different ultrasonic intensities, including the lag phase duration, t_{1/2}, and t_{growth}, where t_{1/2} represents the time required to reach half of the maximum ThT fluorescence intensity, and t_{growth} represents the duration of the exponential phase during the fibrillization process. FIG. 3C shows the maximum ThT fluorescence intensity of pea protein-based amyloid fibrils treated at t0.1 under different ultrasonic intensities.

The results indicated that the t0.1 20% and t0.1 30% groups exhibited distinctive kinetic characteristics. Specifically, in the initial stage of their growth curves, a longer lag phase was observed, followed by a significant increase in the growth rate upon entering the exponential phase, particularly in the t0.1 30% group. This phenomenon was attributed to that the acid hydrolysis and nucleation process occurred during the lag phase, during which protein molecules were initially assembled into oligomers; these protein oligomers acted as templates for fibril growth and rapidly elongated during the exponential phase. For the samples treated with 20% and 30% ultrasonic intensity, the ultrasonic treatment applied at the end of the lag phase may have induced partial disassembly of oligomers, requiring additional time for elongation before entering the exponential phase, thereby prolonging the time required for nucleation and the lag phase. However, the fragmented oligomers provided more active ends during subsequent fibril growth, significantly accelerating the growth rate during the logarithmic growth phase. As shown in FIG. 3C, no significant differences in maximum ThT fluorescence intensity were observed between the control and groups subjected to ultrasonic treatment at t0.1, indicating that ultrasonic treatment at t0.1 had minimal effect on fibril conversion efficiency.

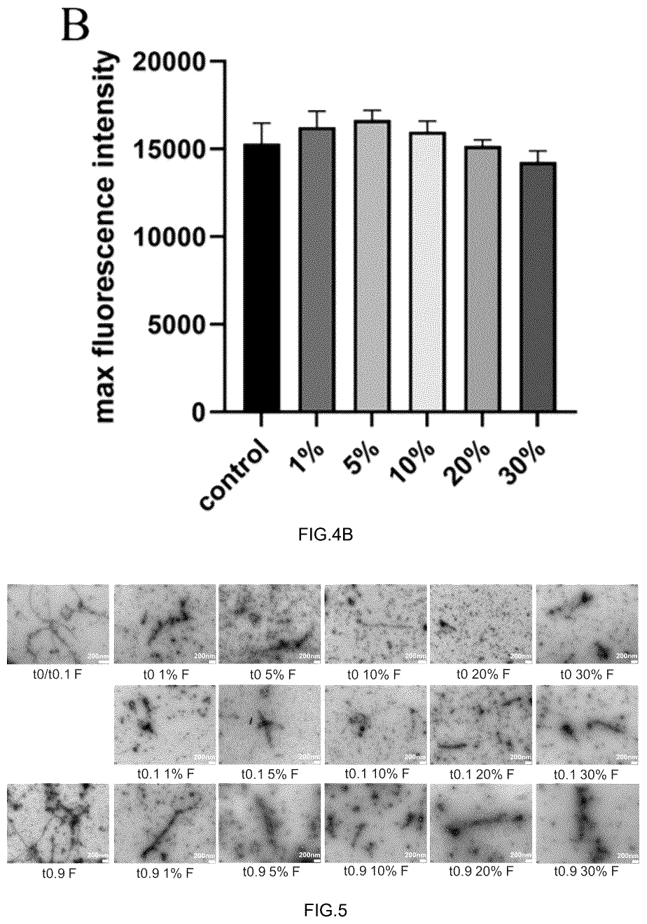

FIGS. 4A-4B show the growth kinetics test results of pea protein-based amyloid fibrils under ultrasonic treatment at t0.9. FIG. 4A shows the normalized growth kinetics curves of pea protein-based amyloid fibrils under ultrasonic treatment at t0.9. FIG. 4B shows the maximum Thioflavin T (ThT) fluorescence intensity of pea protein-based amyloid fibrils treated at t0.9 under different ultrasonic intensities.

The results indicated that, as the fibrillization reaction had entered the saturation stage, the differences in fibril growth kinetics among these samples after ultrasonic treatment were small. For the t0.9 1% sample, the fibril growth rate was slightly accelerated compared with the control, and the maximum ThT fluorescence intensity was slightly increased (P>0.05), suggesting a modest improvement in fibril conversion. This phenomenon was attributed to partial fragmentation of the fibrils caused by the ultrasonic treatment; these fragmented fibril pieces served as nucleation templates, thereby moderately accelerating the self-assembly process. For the other samples in the t0.9 group, as the ultrasonic intensity increased further, more fibril fragments were generated, which increased the number of active fibril ends during the nucleation-growth process; however, the concentration of free proteins available for fibril growth was low, resulting in a reduction in the final fibril growth rate. Therefore, the optimization of ultrasonic intensity is required to balance the generation of fibril fragments and the concentration of free proteins, to maximize the growth efficiency of pea protein-based amyloid fibrils.

### 2. Morphological Analysis of Pea Protein-Based Amyloid Fibrils

To quantitatively analyze the effect of ultrasonic treatment on the morphology of pea protein-based amyloid fibrils, Transmission Electron Microscopy (TEM) images of the fibrils were analyzed using FiberApp. The fibril samples were diluted with an acidic aqueous solution (pH 2.0) to a protein concentration of 0.1 mg/mL. The diluted samples were dropped onto 300-mesh copper grids coated with carbon support films, followed by staining with 5 μL of 2% (w/v) phosphotungstic acid for 30 s. After air drying, the samples were observed at a voltage of 80 KV, and TEM images were captured at 3000× magnification. At least three representative images were selected for each sample, and each selected sample contained at least 100 individual fibrils. FiberApp was used to measure the contour length (Lc) and persistence length (Lp) of each fibril to generate the contour length distribution. Fibrils were classified based on the relationship between Lc and Lp: when the order of magnitude of Lc was smaller than that of Lp, the fibril was defined as rigid; when Lc and Lp were of the same order of magnitude, the fibril was defined as semi-flexible; and when the order of magnitude of Lc was greater than that of Lp, the fibril was defined as flexible. Specifically, the following formula was applied:

$$M = int ( {log}_{10} ( {L}_{c} ) ) - int ( {log}_{10} ( {L}_{p} ) )$$

where M<0 indicated rigid fibrils, M=0 indicated semi-flexible fibrils, and M>0 indicated flexible fibrils.

Based on this classification, the proportions of rigid, semi-flexible, and flexible fibrils were determined.

FIG. 5 shows representative TEM images of the pea protein-based amyloid fibril samples. In FIG. 5, t0 represents the start of fibrillization (0 h), t0.1 represents the end of the lag phase (5 h), and t0.9 represents the end of the exponential phase (26.5 h). t0 (1-30%) F, t0.1 (1-30%) F, and t0.9 (1-30%) F represent the fibril samples formed under ultrasonic treatment at t0, t0.1, and t0.9, respectively. The results showed that all samples exhibited diverse morphologies, including rigid, semi-flexible, and flexible fibrils.

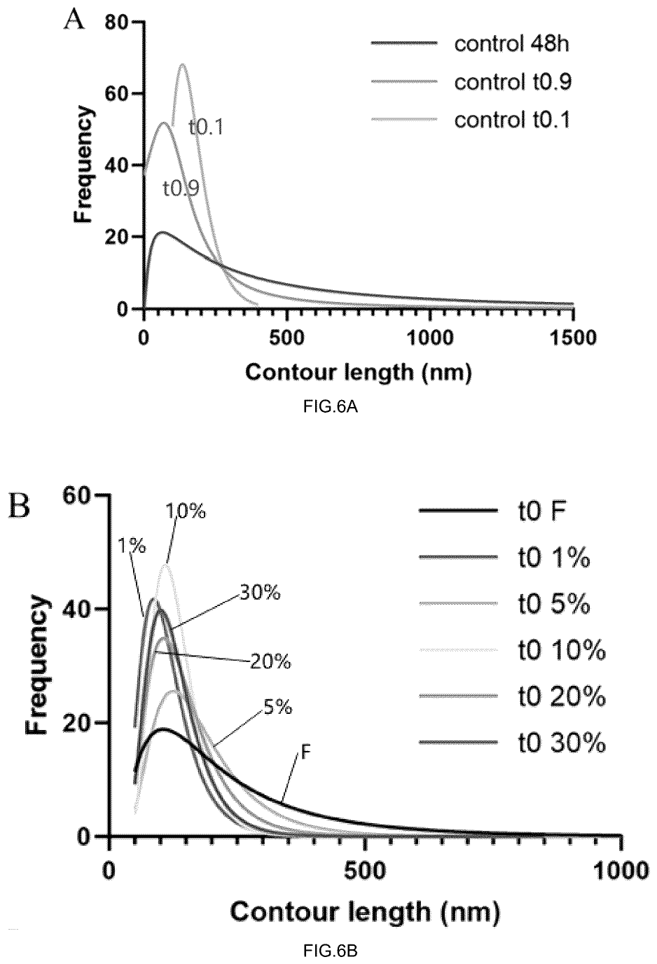

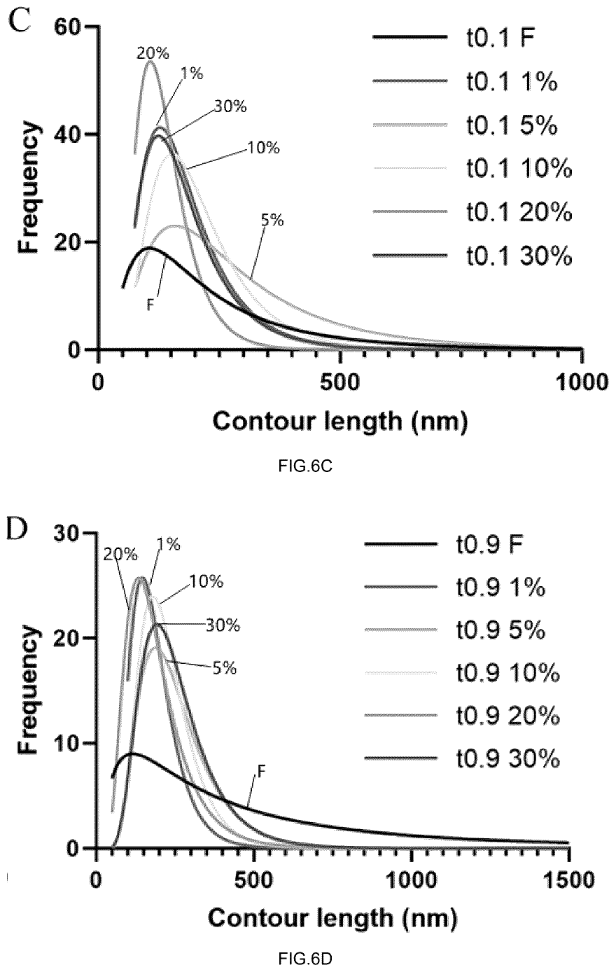

FIGS. 6A-6D show the contour length distributions of pea protein-based amyloid fibrils under ultrasonic treatment. FIG. 6A shows the contour length distribution of fibril samples with different heating durations. FIG. 6B shows the contour length distribution of fibril samples that were subjected to ultrasonic treatment at the beginning of the lag phase (0 h) and subsequently incubated. FIG. 6C shows the contour length distribution of fibrils in samples that were subjected to ultrasonic treatment at the end of the lag phase (5 h) and subsequently incubated. FIG. 6D shows the contour length distribution of fibrils in samples that were subjected to ultrasonic treatment at the end of the exponential phase (26.5 h) and subsequently incubated.

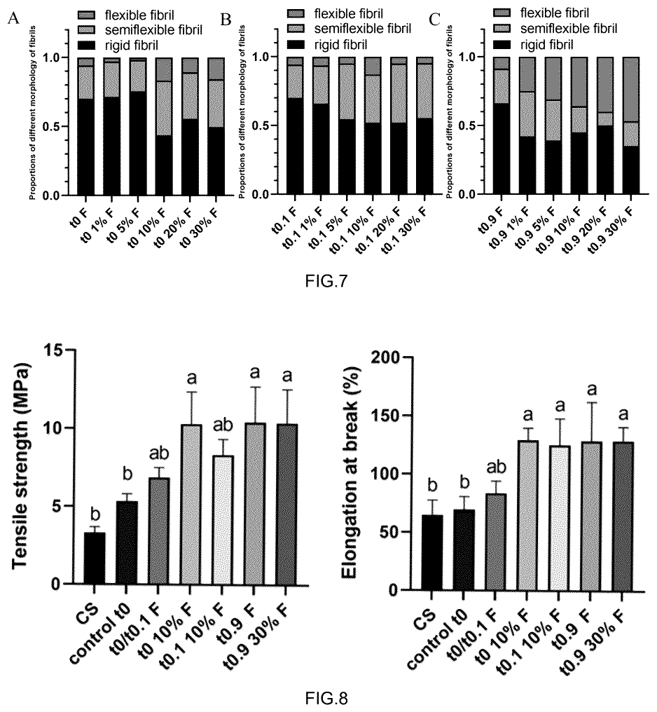

FIG. 7 is a statistical diagram of the proportions of flexible, semi-flexible, and rigid fibrils among pea protein-based amyloid fibrils under ultrasonic treatment at t0, t0.1, and t0.9. In FIG. 7, panel A presents the proportions of rigid, semi-flexible, and flexible fibrils in samples that were subjected to ultrasonic treatment at the beginning of the lag phase (0 h) and subsequently incubated; panel B shows the proportions of rigid, semi-flexible, and flexible fibrils in samples that were subjected to ultrasonic treatment at the end of the lag phase (5 h) and subsequently incubated; panel C shows the proportions of rigid, semi-flexible, and flexible fibrils in samples that were subjected to ultrasonic treatment at the end of the exponential phase (26.5 h) and subsequently incubated. The detailed results were listed in Table 2.

TABLE 2
Proportions of rigid, semi-flexible, and flexible
fibrils in pea protein-based amyloid fibrils

| | | Proportion | Proportion |
|---|---|---|---|
| | Proportion | of Semi- | of |
| Sample | of Rigid | flexible | Flexible |
| Type | Fibrils | Fibrils | Fibrils |
| t0 F | 70.00% | 24.17% | 5.83% |
| t0 1% F | 71.43% | 25.51% | 3.06% |
| t0 5% F | 75.49% | 22.55% | 1.96% |
| t0 10% F | 43.56% | 39.60% | 16.83% |
| t0 20% F | 55.45% | 33.66% | 10.89% |
| t0 30% F | 49.51% | 34.65% | 15.84% |
| t0.1 F | 70.00% | 24.17% | 5.83% |
| t0.1 1% F | 65.74% | 27.78% | 6.48% |
| t0.1 5% F | 54.46% | 40.59% | 4.95% |
| t0.1 10% F | 52.00% | 35.00% | 13.00% |
| t0.1 20% F | 52.00% | 43.00% | 5.00% |
| t0.1 30% F | 55.24% | 40.00% | 4.76% |
| t0.9 F | 66.00% | 25.33% | 8.67% |
| t0.9 1% F | 42.00% | 33.00% | 25.00% |
| t0.9 5% F | 39.00% | 30.00% | 31.00% |
| t0.9 10% F | 45.00% | 19.00% | 36.00% |
| t0.9 20% F | 50.00% | 10.00% | 40.00% |
| t0.9 30% F | 35.00% | 18.00% | 47.00% |

The results demonstrated that the contour length of pea protein-based amyloid fibrils increased with reaction time during the fibrillization process. However, the fibril contour length was significantly shortened by ultrasonic treatment at to, t0.1, and t0.9. This was attributed to fibril fragmentation caused by ultrasonic treatment, which provided more active ends for elongation but limited the overall increase in the contour length. As shown in FIG. 7, ultrasonic treatment also significantly altered the proportion of fibrils with different morphologies. For the samples treated at t0, the ultrasonic treatment with high power density substantially decreased the proportion of rigid fibrils while increasing the proportion of flexible and semi-flexible fibrils. Specifically, for t0 10% F, compared with to/t0.1 F, the proportion of flexible fibrils increased from 5.83% to 16.83%, and the proportion of semi-flexible fibrils increased from 24.17% to 39.60%. In contrast, at lower ultrasonic power density, the proportion of rigid fibrils slightly increased. For all ultrasonic intensities in the t0.1 group, the proportion of rigid fibrils was significantly reduced, while the proportion of flexible fibrils remained relatively low. Exceptions included t0.1 10% F, where the proportion of flexible fibrils increased from 5.83% to 13.00%, and the proportion of semi-flexible fibrils increased from 24.17% to 35.00%, compared with t0/t0.1 F. In the t0.9 group, as the power density of the ultrasonic treatment increased, the proportion of flexible fibrils increased significantly. For t0.9 30% F, the proportion of flexible fibrils increased from 8.67% to 47.00%, while the proportion of rigid fibrils decreased from 66.00% to 35.00%, compared with t0.9 F.

### Example 2

Preparation of pea protein-based amyloid fibril-chitosan composite films including the steps as follows.

Chitosan (purchased from Shanghai Aladdin Biochemical Technology Co., Ltd.) was mixed with acetic acid solution (with a volume concentration of 2% v/v) at a ratio of 3 g:100 mL. The mixture was stirred until the chitosan was completely dissolved. The pH of the resulting solution was adjusted to 2.0 using 3 M HCl to obtain a 3% (w/v) chitosan solution.

The pea protein-based amyloid fibrils prepared in Example 1 were dispersed in water at a ratio of 1 g:100 mL to obtain a 1% (w/v) fibril dispersion.

Then, 15 mL of the fibril dispersion was mixed with 15 mL of the chitosan solution, and 0.5 mL of glycerol was added. The mixture was incubated in a 60° C. water bath for 30 min under stirring and subsequently degassed in an ultrasonic cleaner for 30 min to obtain a film-forming solution. The 30 mL film-forming solution was poured into a 9 cm-diameter petri dish and dried in a 50° C. drying oven for 18 h, then equilibrated in a desiccator for 24 h to obtain the pea protein-based amyloid fibril-chitosan composite film.

A control group and a blank group were prepared as follows. For the control group, 15 mL of 1% (w/v) pea protein isolate solution was used to replace the fibril dispersion, while for the blank group, 15 mL of acidic aqueous solution (pH 2.0) was used to replace the fibril dispersion. All other procedures were the same to those in Example 2.

The composite films prepared using pea protein-based amyloid fibrils were designated according to the naming convention of the fibrils themselves, and were denoted as t0/t0.1 F, t0 10% F, t0.1 10% F, t0.9 F, and t0.9 30% F. The composite film prepared using pea protein isolate was denoted as control to, and the composite film prepared using ultrapure water was denoted as CS.

The thicknesses of the composite films were summarized in Table 3.

TABLE 3
Thickness of Composite Films

| Composite Film | Thickness |
|---|---|
| Sample Type | (mm) |
| CS | 0.153 ± 0.001 |
| control t0 | 0.157 ± 0.026 |
| t0/t0.1 F | 0.151 ± 0.021 |
| t0 10% F | 0.149 ± 0.007 |
| t0.1 10% F | 0.150 ± 0.014 |
| t0.9 F | 0.146 ± 0.010 |
| t0.9 30% F | 0.146 ± 0.012 |

### Test Example 2: Evaluation of Mechanical Properties of Composite Films

The film samples were cut into strips measuring 60 mm×10 mm, and their mechanical properties were measured using a texture analyzer. The initial gauge length (i.e., the initial distance between the grips) was set to 30 mm, and the stretching speed was set to 50 mm/min until the film samples fractured. The tensile strength (TS) and elongation at break (E) of the films were calculated using the following formulas:

$$TS ( MPa ) = \frac{F}{a \times b}$$

$$E ( \% ) = \frac{L - {L}_{0}}{{L}_{0}}$$

where F is the maximum force at break (N), a is the width of the film (mm), b is the thickness of the film (mm), L is the length of the film at break (mm), and Lo is the initial length of the film (mm).

FIG. 8 shows the results of tensile strength and elongation at break of the composite films, where the left panel presents tensile strength and the right panel shows elongation at break. The results indicated that the CS (film prepared from only chitosan) group exhibited poor tensile strength and elongation at break, measuring 3.32 MPa and 64.93%, respectively, indicating that when chitosan alone was used as the film-forming matrix, the mechanical properties were inferior. After pea protein isolate was added to the chitosan film system (control to group), the tensile strength and elongation at break of the composite film were slightly improved compared to the CS group, although no significant differences were observed. This is because the pea protein isolate in its native conformation may generate weak intermolecular interactions with chitosan molecules, but cannot form a stacked network structure.

After pea protein-based amyloid fibrils were incorporated into the chitosan film system, the tensile strength and elongation at break of the composite films were significantly enhanced. Specifically, the t0 10% F, t0.1 10% F, control 48 h, and t0.9 30% F groups exhibited significant improvements compared to CS, while the t0/t0.1 F group did not show a significant improvement. Further analysis indicated that when the stiffness/flexibility of fibrils is comparable, the contour length of the fibril samples was positively correlated with the mechanical performance of the composite film. This conclusion was supported by the fact that the t0/t0.1 F and control 48 h groups both contained pea protein-based amyloid fibrils that had not undergone ultrasonic treatment, although to/t0.1 F group underwent a shorter heating duration than control 48 h group and consequently produced fibril sample with a shorter contour length (as shown in FIG. 6), the stiffness/flexibility of the fibril samples in the two groups were statistically similar. This might be because fibrils with longer contour lengths could form networks with more crosslinking points, and the mechanical properties of these networks were positively correlated with the rigidity and flexibility of macromolecules and the number of crosslinking points. Therefore, the fibrils with longer contour length exhibited superior mechanical properties.

Moreover, the mechanical properties of the t0/t0.1 F, to 10% F, and t0.1 10% F groups were compared, the results indicated that the t0 10% F and t0.1 10% F groups exhibited superior mechanical properties relative to the to/t0.1 F group. This improvement may be attributed to the ultrasonic treatment applied to the t0 10% F and t0.1 10% F groups. After ultrasonic treatment, the contour length of the fibrils decreased, but the proportion of flexible and semi-flexible fibrils significantly increased (as shown in FIG. 7). This indicated that the rigidity and flexibility of fibrils significantly influenced the mechanical properties of the composite films. Specifically, a higher proportion of flexible or semi-flexible fibrils promoted molecular entanglement, increasing the number of network crosslinking points and thereby enhancing the mechanical properties of the composite films.

FIG. 9 shows the correlation between fibril rigidity and flexibility and the mechanical properties of the films. In FIG. 9, the symbols “+” and “−” denote positive and negative correlations, respectively. Specifically, a correlation analysis was performed between the stiffness/flexibility of the fibril samples and the tensile strength and elongation at break of the composite films. The results showed that the proportion of rigid fibrils is negatively correlated with both the tensile strength and elongation at break, whereas the proportions of semi-flexible and flexible fibrils are positively correlated with both the tensile strength and elongation at break. Compared to the to/t0.1 F group, the t0 10% F and t0.1 10% F groups that were subjected to ultrasonic treatment contained higher proportions of flexible fibrils and lower proportions of rigid fibrils; consequently, the composite films prepared from these two groups demonstrated enhanced mechanical strength.

### Example 3

Preparation of composite coating preservative includes the steps as follows.

The film-forming solution was prepared according to the method described in Example 2. Then, 375 μL of EGCG solution (20 mg/mL, pH 2.0) was added to the film-forming solution, such that the mass ratio of pea protein-based amyloid fibrils to EGCG in the film-forming solution was 20:1, yielding the composite coating preservative.

### Test Example 3

### 1. Evaluation for In Vitro Antioxidant Activity

The antioxidant activity of pea protein isolate and pea protein-based amyloid fibril samples was evaluated using the DPPH assay. DPPH was first dissolved in anhydrous ethanol to obtain a 0.1 mM DPPH working solution. Then, 200 μL of pea protein isolate dispersion (20 mg/mL, pH 2.0, control group) or pea protein-based amyloid fibril dispersion (20 mg/mL, pH 2.0, experimental group) was mixed with 800 μL of DPPH working solution. For the blank group, 200 μL of acidic aqueous solution (pH 2.0) was mixed with 800 μL of DPPH working solution. The resulting mixtures were incubated in the dark at 25° C. for 30 min, and the absorbance of each sample at 517 nm was measured using a microplate reader. The DPPH radical scavenging activity of the samples was calculated as follows:

$$DPPH radical scavenging activity = \frac{{A}_{0} - {A}_{1}}{{A}_{0}} \times 100 \%$$

where A₀ is the absorbance of the blank group, and A₁ is the absorbance of the sample.

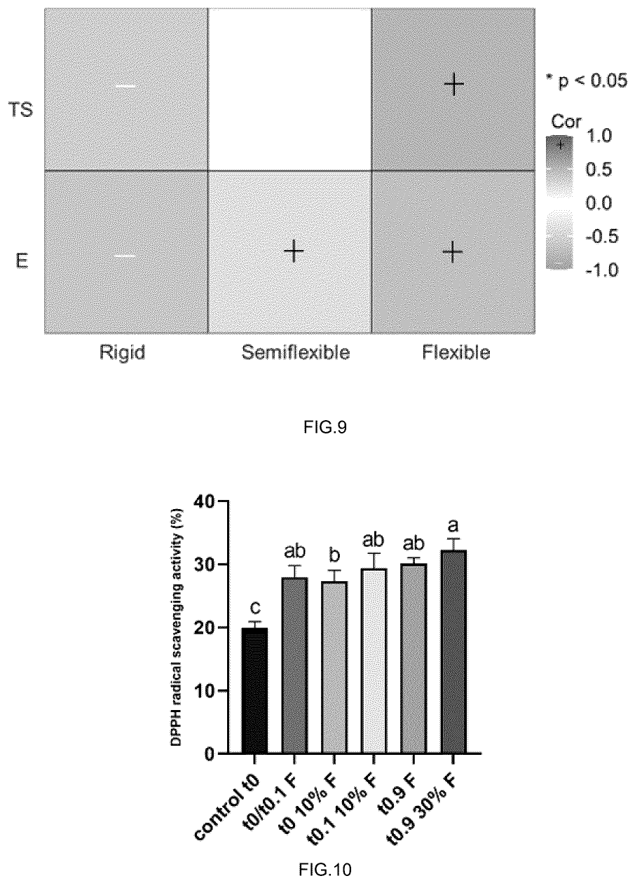

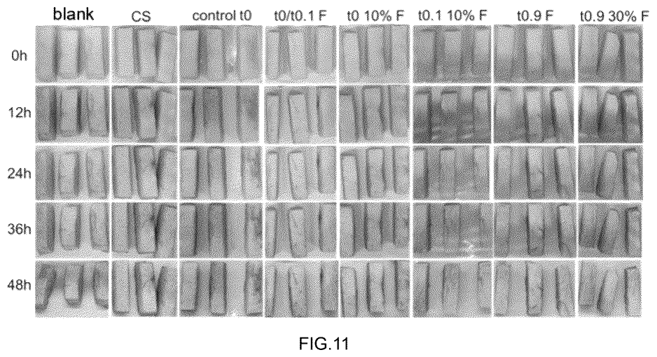

FIG. 10 shows the DPPH radical scavenging activity results for pea protein isolate and pea protein-based amyloid fibrils. The results indicated that after pea protein isolate was converted into pea protein-based amyloid fibrils, its DPPH radical scavenging activity was significantly enhanced. This improvement is ascribed to the exposure of more cysteine residues upon fibril formation. These cysteine residues, which were originally buried within the pea protein isolate in its native structure, possess inherent antioxidant activity. Among all fibril samples, the t0.9 F and t0.9 30% F groups exhibited higher DPPH scavenging activities, measuring 30.14±0.88% and 32.23±1.86%, respectively. This suggested that longer incubation of pea protein-based amyloid fibrils led to greater exposure of cysteine residues. The enhanced antioxidant activity of pea protein-based amyloid fibrils suggested their potential for preserving fruits prone to browning.

### 2. Evaluation of Composite Coating Preservative for the Preservation of Fresh-Cut Apples

Fresh apples were washed, peeled, and cut into uniform strips. The cut apple strips were immediately immersed in the composite coating preservative for 30 s, taken out, and then stored at 25° C. with 75% relative humidity. Photographs were taken every 12 h. After 48 h, the color of all apple samples was measured.

A blank group and a control group were included. The fresh-cut apple samples of the blank group were left uncoated. The fresh-cut apple samples of the control group (CS) were coated with a chitosan-based coating preservative. For the control group, the pea protein-based amyloid fibril dispersion was replaced by 15 mL of acidic aqueous solution (pH 2.0) during the preparation of the composite coating preservative.

FIG. 11 shows the test results of fresh-cut apples. The results indicated that after the application of the coating preservative, the shape of the fresh-cut apples in control group and experiment group remained relatively consistent before and after storage, and browning was suppressed to varying degrees. After 12 h of storage, noticeable browning was observed in the blank group. However, browning was inhibited in all groups containing pea protein-based amyloid fibrils, showing minimal difference compared to the samples at 0 h of storage. The following trend became more pronounced with extended storage time: the CS group performed better than the blank group, the control to group slightly outperformed the CS group, and groups containing pea protein-based amyloid fibrils showed superior browning inhibition compared to all other groups. This corresponded with the antioxidant activities of the samples. Moreover, pea protein-based amyloid fibrils exhibited stronger binding with EGCG than pea protein isolate, thereby providing enhanced anti-browning capacity.

Table 4 summarizes the CIEL*a*b* values of fresh-cut apples after 48 h of storage. L* represented the lightness of the sample, a* represented the red-green coordinate (redness/greenness), b* represented the yellow-blue coordinate (yellowness/blueness), where a higher b* indicated a more yellow appearance, and ΔE represented the color difference relative to the standard. The b* values for samples containing pea protein isolate or pea protein-based amyloid fibrils were ranked as follows: control to >t0 10% F>t0/t0.1 F>t0.9 F>t0.9 30% F>t0.1 10% F. This ranking aligned with the antioxidant activity trend of the corresponding pea protein isolate and pea protein-based amyloid fibril samples (as shown in FIG. 10), indicating that the pea protein-based amyloid fibril samples with higher antioxidant activity can more effectively inhibited browning of fresh-cut apples. The results further indicated that pea protein-based amyloid fibrils with longer incubation times exhibited stronger anti-browning capability (t0/t0.1 F<10.9 F, P>0.05), as longer-incubated fibrils had greater contour lengths, allowing for higher EGCG loading and providing a sustained-release effect during storage.

Furthermore, differences in fibril morphology also influenced anti-browning performance. Fibrils obtained under ultrasonic treatment, containing higher proportions of flexible fibrils (t0.1 10% F and t0.9 30% F), exhibited superior browning inhibition compared to their respective control fibril groups, that is, t0.1 10% F>t0/t0.1 F, t0.9 30% F>10.9 F. Notably, the t0.1 10% F group showed significant improvement compared to its control, indicating that samples with higher proportions of flexible and semi-flexible fibrils could load more EGCG, and the denser network structures formed in the flexible and semi-flexible fibrils enhanced the sustained-release effect of EGCG.

TABLE 4
CIEL*a*b* Values of Fresh-Cut Apples after
48 h of Storage

| Sam- | | | | |
|---|---|---|---|---|
| ple | | | | |
| Type | L* | a* | b* | ΔΕ |
| Blank | 81.01 ± | 13.95 ± | 41.61 ± | 92.15 ± |
| | 2.47^{ab} | 1.37^{ª} | 0.58^{ª} | 2.20ª |
| CS | 70.83 ± | 11.78 ± | 38.07 ± | 81.34 ± |
| | 7.22^{b} | 0.77^{ab} | 0.64^{b} | 5.99^{b} |
| Con- | 75.29 ± | 10.13 ± | 35.69 ± | 84.01 ± |
| trol | 4.35^{b} | 1.09^{b} | 1.76^{b} | 3.17^{ab} |
| t0 | | | | |
| t/ | 78.86 ± | 9.05 ± | 34.19 ± | 86.44 ± |
| t0.1 F | 4.36^{ab} | 0.71^{bc} | 1.06^{b} | 4.12^{ab} |
| t0 | 81.44 ± | 9.99 ± | 34.96 ± | 89.18 ± |
| 10% F | 0.81^{ab} | 0.50^{bc} | 0.30^{b} | 0.82^{ab} |
| t0.1 | 88.15 ± | 5.76 ± | 27.90 ± | 92.64 ± |
| 10% F | 0.91^{ª} | 0.20^{c} | 0.95^{c} | 1.16ª |
| t0.9 F | 86.23 ± | 7.39 ± | 31.18 ± | 92.02 ± |
| | 1.17^{ª} | 1.54^{c} | 1.95^{bc} | 0.54ª |
| t0.9 | 86.98 ± | 6.11 ± | 28.05 ± | 91.59 ± |
| 30% F | 2.31^{ª} | 0.10^{c} | 1.53^{c} | 2.65ª |

The above-described embodiments represented preferred implementations of the present disclosure. It should be noted that persons skilled in the art could make various modifications and refinements without departing from the principles of the invention, and such modifications and refinements should also be considered within the scope of the present disclosure.

What is claimed is:

1. A method for preparing plant-based amyloid fibrils, comprising:
1) mixing a plant protein isolate with water to obtain an aqueous solution of the plant protein isolate, wherein the plant protein isolate comprises pea protein isolate;
2) adjusting the pH of the aqueous solution of the plant protein isolate to 1.5-2.5, followed by solid-liquid separation to obtain a liquid material containing the plant protein isolate; and
3) subjecting the liquid material containing the plant protein isolate to a fibrillization treatment to obtain the plant-based amyloid fibrils;
wherein the fibrillization treatment sequentially comprises a first incubation treatment, an ultrasonic treatment, and a second incubation treatment; and both the first and second incubation treatments are performed under non-ultrasonic conditions;
wherein temperatures of the first and second incubation treatments are independently in a range of 75-85° C.; a total duration of the first and second incubation treatments is in a range of 45-50 h; and the first and second incubation treatments are performed under stirring;
wherein a temperature of the ultrasonic treatment is in a range of 0-4° C.; a total duration of the ultrasonic treatment is in a range of 1-3 min; the ultrasonic treatment is performed with an interval of 1-3 seconds after every 1-3 seconds of ultrasonic irradiation; and
wherein the fibrillization treatment comprises any one of the following:
1) a duration of the first incubation treatment is 0 h, and an ultrasonic power density of the ultrasonic treatment is in a range of 2.6-4 W/mL;
2) the duration of the first incubation treatment is in a range of 4-5 h, and the ultrasonic power density of the ultrasonic treatment is in a range of 3-4 W/mL; and
3) the duration of the first incubation treatment is in a range of 25-26.5 h, and the ultrasonic power density of the ultrasonic treatment is in a range of 2-2.2 W/mL.

2. The method of claim 1, wherein a mass-to-volume ratio of the plant protein isolate to water is in a range of 1-3 g:100 mL.

3. Plant-based amyloid fibrils prepared by the method of claim 1, comprising flexible fibrils, semi-flexible fibrils, and rigid fibrils, wherein a proportion of flexible fibrils in the plant-based amyloid fibrils is in a range of 4-50%, a proportion of semi-flexible fibrils in the plant-based amyloid fibrils is in a range of 10-45%, and a proportion of rigid fibrils in the plant-based amyloid fibrils is in a range of 35-65.8%.

4. A composite film, comprising a chitosan matrix and the plant-based amyloid fibrils of claim 3, wherein the plant-based amyloid fibrils are distributed in the chitosan matrix.

5. A composite coating preservative, comprising chitosan, the plant-based amyloid fibrils of claim 3, epigallocatechin gallate (EGCG), and a solvent.

6. Plant-based amyloid fibrils prepared by the method of claim 2, comprising flexible fibrils, semi-flexible fibrils, and rigid fibrils, wherein a proportion of flexible fibrils in the plant-based amyloid fibrils is in a range of 4-50%, a proportion of semi-flexible fibrils in the plant-based amyloid fibrils is in a range of 10-45%, and a proportion of rigid fibrils in the plant-based amyloid fibrils is in a range of 35-65.8%.

7. A composite film, comprising a chitosan matrix and the plant-based amyloid fibrils of claim 6, wherein the plant-based amyloid fibrils are distributed in the chitosan matrix.

8. A composite coating preservative, comprising chitosan, the plant-based amyloid fibrils of claim 6, epigallocatechin gallate (EGCG), and a solvent.
